(19) United States
(12) Patent Application Publication
(10) Pub. No.: US 2021/0022799 A1
(43) Pub. Date: Jan. 28, 2021

(54) SURGICAL INSTRUMENT INCLUDING A PIEZO-ELEMENT FOR ADJUSTING A POSITION OF A MECHANICAL COMPONENT OF THE SURGICAL INSTRUMENT

(71) Applicant: Covidien LP, Mansfield, MA (US)

(72) Inventors: Gregory W. Fischvogt, Reno, NV (US); Michael D. Morris, Thornton, CO (US)

(21) Appl. No.: 16/936,657

(22) Filed: Jul. 23, 2020

Related U.S. Application Data
(60) Provisional application No. 62/878,411, filed on Jul. 25, 2019.

(51) Int. Cl. A61B 18/14

(52) U.S. Cl. CPC A61B 18/1445; A61B 2017/00725

(57) ABSTRACT

A surgical instrument includes a handle housing, a handle operably coupled to the handle housing, an outer shaft, and an inner shaft. The outer shaft extends distally from the handle housing, and the inner shaft is axially disposed within the outer shaft. At least one of the outer shaft or the inner shaft is selectively movable relative to the other along a longitudinal axis in response to actuation of the handle. A piezoelectric actuator is coupled to the outer shaft or the inner shaft and is configured to adjust an axial position of the outer shaft and/or the inner shaft.

### CROSS-REFERENCE TO RELATED APPLICATIONS

This application claims the benefit of and priority to U.S. Provisional Patent Application Ser. No. 62/878,411 filed Jul. 25, 2019, the entire disclosure of which is incorporated by reference herein.

### FIELD

The present technology is generally related to the field of surgical instruments, and more particularly to a surgical instrument with a piezo-element for adjusting a position of a mechanical component of the surgical instrument.

### BACKGROUND

Instruments such as electrosurgical forceps are commonly used in open and endoscopic surgical procedures to coagulate, cauterize, and seal tissue. Such forceps typically include a pair of jaws that can be controlled by a surgeon to grasp targeted tissue, such as, e.g., a blood vessel. The jaws may be approximated to apply a mechanical clamping force to the tissue, and are associated with at least one electrode to permit the delivery of electrosurgical energy to the tissue.

Both the pressure applied by the jaws and the gap distance between the jaws influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied, the tissue may have a tendency to move before an adequate seal can be generated. There is continued interest in improving the pressure and gap distance of instruments such as electrosurgical forceps.

### SUMMARY

The techniques of this disclosure generally relate to a surgical instrument with an adjustable mechanical output.

In one aspect, the disclosure provides a surgical instrument including a handle housing, a handle operably coupled to the handle housing, an outer shaft, and an inner shaft. The outer shaft extends distally from the handle housing, and the inner shaft is axially disposed within the outer shaft. The inner shaft has a cam pin mechanically coupled to a distal end portion thereof. At least one of the outer shaft or the inner shaft is selectively movable relative to the other along a longitudinal axis in response to actuation of the handle. The surgical instrument further includes a piezoelectric actuator coupled to the outer shaft or the inner shaft. The piezoelectric actuator is configured to adjust a distance between a distal end of the outer shaft and a distal end of the inner shaft.

In aspects, the piezoelectric actuator may be a programmable piezo-based shim.

In aspects, the piezoelectric actuator may be disposed between a proximal end of the inner shaft and a portion of the housing, such that an actuation of the piezoelectric actuator adjusts an axial location of the proximal end of the inner shaft relative to the portion of the housing.

In aspects, the proximal end of the inner shaft may be fixed to the piezoelectric actuator and the outer shaft may be configured to move relative to the inner shaft along the longitudinal axis in response to actuation of the handle.

In aspects, the piezoelectric actuator may be disposed between and interconnect a proximal end portion of the inner shaft and the distal end portion of the inner shaft.

In aspects, the piezoelectric actuator may be disposed between and interconnect a proximal end portion of the outer shaft and a distal end portion of the outer shaft.

In aspects, the surgical instrument may further include an end effector including a pair of opposing first and second jaw members. The first and second jaw members may be operably coupled about a common pivot, such that at least one of the jaw members is movable relative to the other jaw member from a first position in which the jaw members are disposed in spaced relation to one another to a second position, in which the jaw members cooperate to grasp tissue therebetween. At least one of the first and second jaw members may define a camming slot configured to engage the cam pin to move the jaw member between the first position and the second position upon relative longitudinal movement between the inner and outer shafts.

In aspects, the surgical instrument may further include a switch supported by the handle housing. The switch may be configured to be engaged by the handle to initiate delivery of electrosurgical energy from an electrosurgical energy source to the end effector to treat tissue.

In another aspect, the disclosure provides a method of calibrating a surgical instrument. The method includes sending a signal representative of a calibration value from the surgical instrument to a generator. The generator is electromechanically coupled to the surgical instrument. The method further includes causing a piezoelectric actuator disposed within the surgical instrument to move a mechanical component of the surgical instrument a distance corresponding to the calibration value to adjust a mechanical output of the surgical instrument.

In aspects, the mechanical component of the surgical instrument may be an inner shaft or an outer shaft. At least one of the outer shaft or the inner shaft may be selectively movable relative to the other in response to actuation of a handle of the surgical instrument to move an end effector between an open and closed configuration.

In aspects, moving the mechanical component may include changing a distance between a distal end of the outer shaft and a distal end of the inner shaft.

In aspects, the piezoelectric actuator may be coupled to a proximal end of the inner shaft, such that the actuation of the piezoelectric actuator adjusts an axial location of the proximal end of the inner shaft.

In aspects, the mechanical output may be a force applied by the end effector to tissue upon moving the end effector to the closed configuration.

In aspects, the mechanical output may be a gap defined between first and second jaw members of the end effector upon moving the end effector to the closed configuration.

In aspects, the method may further include detecting an electrical short between jaw members of the end effector and actuating the piezoelectric actuator to increase a gap defined between the jaw members in response to detecting the electrical short.

In aspects, actuating the piezoelectric actuator may include delivering electricity from the generator to the piezoelectric actuator to alter a shape of the piezoelectric actuator.

In another aspect, the disclosure provides an electrosurgical system for performing electrosurgery. The electrosurgical system includes an electrosurgical generator configured to provide electrosurgical energy and an electrosurgical instrument. The electrosurgical instrument includes a handle housing, a handle operably coupled to the handle housing, an outer shaft, and an inner shaft. The outer shaft extends distally from the handle housing, and the inner shaft is axially disposed within the outer shaft. At least one of the outer shaft or the inner shaft is selectively movable relative to the other along a longitudinal axis in response to actuation of the handle. The electrosurgical system further includes a piezoelectric actuator coupled to the outer shaft or the inner shaft and is in electrical communication with the generator. The piezoelectric actuator is configured to adjust a distance between a distal end of the outer shaft and a distal end of the inner shaft in response to an electrical signal received from the generator.

In aspects, the piezoelectric actuator may be disposed between a proximal end of the inner shaft and a portion of the housing, such that an actuation of the piezoelectric actuator adjusts an axial location of the proximal end of the inner shaft relative to the portion of the housing.

In aspects, the proximal end of the inner shaft may be fixed to the piezoelectric actuator and the outer shaft may be configured to move relative to the inner shaft along the longitudinal axis in response to actuation of the handle.

In aspects, the surgical instrument may include a pair of opposing first and second jaw members operably coupled about a common pivot. The first and second jaw members may be configured to move between open and closed configurations in response to relative longitudinal movement between the inner and outer shafts. The actuation of the piezoelectric actuator may adjust at least one of a force applied to tissue disposed between the pair of first and second jaw members or a gap defined between the pair of first and second jaw members.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The accompanying drawings, which are incorporated in and constitute a part of this specification, illustrate embodiments of the disclosure and, together with the detailed description of the embodiments given below, serve to explain the principles of the disclosure.

FIG. 1 is a perspective view that illustrates an electrosurgical forceps according to an embodiment of the disclosure including a housing, a shaft assembly, and an end effector;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3 is a perspective view of the end effector and shaft assembly of FIG. 1 with parts separated;

FIG. 4 is a perspective view illustrating a proximal portion of the instrument of FIG. 1 with a portion of the housing removed revealing internal components;

FIG. 5 is a partial, side view of the proximal portion of the instrument of FIG. 1 with the portion of the housing removed;

FIG. 6 is a side view illustrating the proximal portion of the instrument of FIG. 1 including a piezoelectric actuator coupled to an inner shaft of the shaft assembly;

FIG. 7 is a side view illustrating the proximal portion of the instrument of FIG. 1 including a piezoelectric actuator coupled between proximal and distal end portions of the inner shaft;

FIG. 8 is a side view illustrating the proximal portion of the instrument of FIG. 1 including a piezoelectric actuator coupled between proximal and distal end portions of an outer shaft of the shaft assembly; and

FIG. 9 is a side view illustrating the proximal portion of the instrument of FIG. 1 including a piezoelectric actuator coupled between proximal and distal end portions of the outer shaft and a piezoelectric actuator coupled to the inner shaft.

### DETAILED DESCRIPTION

Particular embodiments of the disclosure are described hereinbelow with reference to the accompanying drawings. In the following description, well-known functions or constructions are not described in detail to avoid obscuring the disclosure in unnecessary detail. As used herein, the term “distal” refers to that portion which is further from the user while the term “proximal” refers to that portion which is closer to the user or surgeon.

The surgical instruments provided herein include features that adjust for inherent variations in the manufacture thereof. For example, the surgical instruments may include features that enable adjustment of mechanical output of the surgical instrument during and after using the surgical instrument.

The systems and methods of the disclosure detailed below may be incorporated into different types of surgical configurations or procedures. The particular illustrations and embodiments disclosed herein are merely exemplary and do not limit the scope or applicability of the disclosed technology.

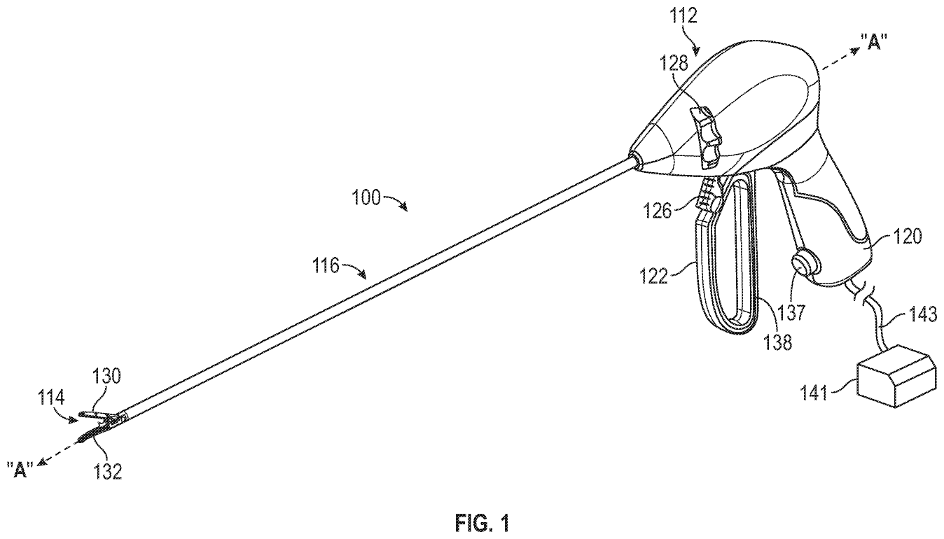

Referring initially to FIG. 1, an embodiment of a surgical instrument, such as, for example, an electrosurgical forceps 100, generally includes a handle housing 112 that supports various actuators thereon for remotely controlling an end effector 114 through an elongated shaft assembly 116. Although this configuration is typically associated with instruments for use in laparoscopic or endoscopic surgical procedures, various aspects of the disclosure may be practiced with traditional open instruments and in connection with certain endoluminal procedures.

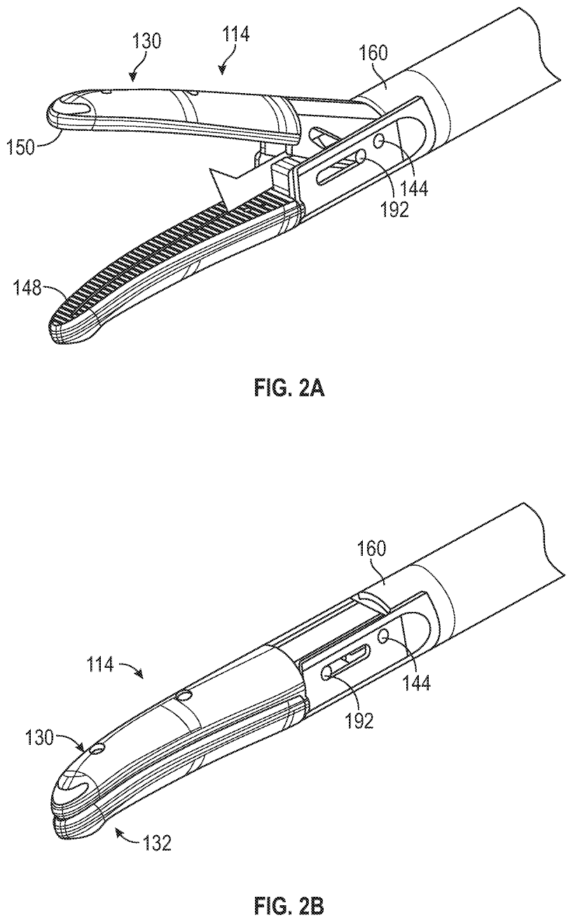

With reference to FIGS. 1-5, to mechanically control the end effector 114, the handle housing 112 supports a stationary handle 120, a movable handle 122, a knife trigger 126, and a rotation knob 128. The movable handle 122 is operable to move the end effector 114 between an open configuration (FIG. 2A) wherein a pair of opposed jaw members 130, 132 are disposed in spaced relation to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration.

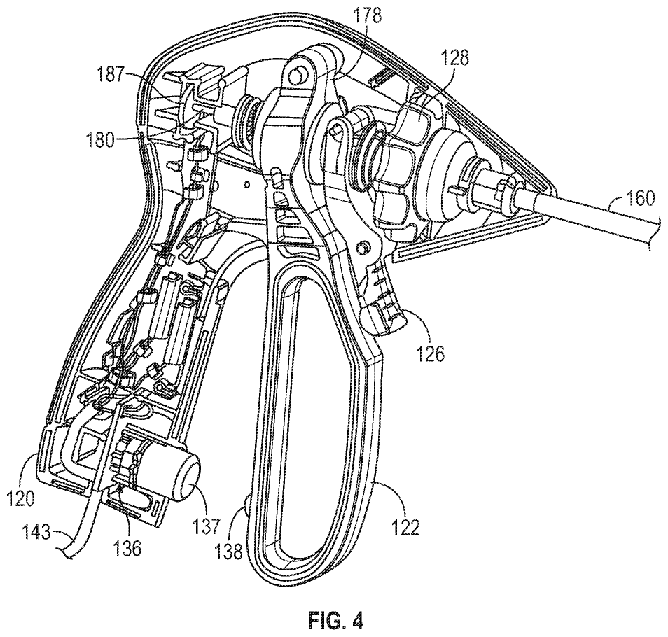

To electrically control the end effector 114, the stationary handle 120 supports a depressible button 137 thereon, which is operable by the user to initiate and terminate the delivery of electrosurgical energy to the end effector 114. More specifically, the depressible button 137 is mechanically coupled to a switch 136 (FIG. 4) disposed within the stationary handle 120 and is engageable by a button activation post 138 extending from a proximal side of the movable handle 122 upon proximal movement of the movable handle 122 to an actuated or proximal position. The switch 136 is in electrical communication with a source of electrosurgical energy such as electrosurgical generator 141 or a battery (not shown) supported within the housing 112.

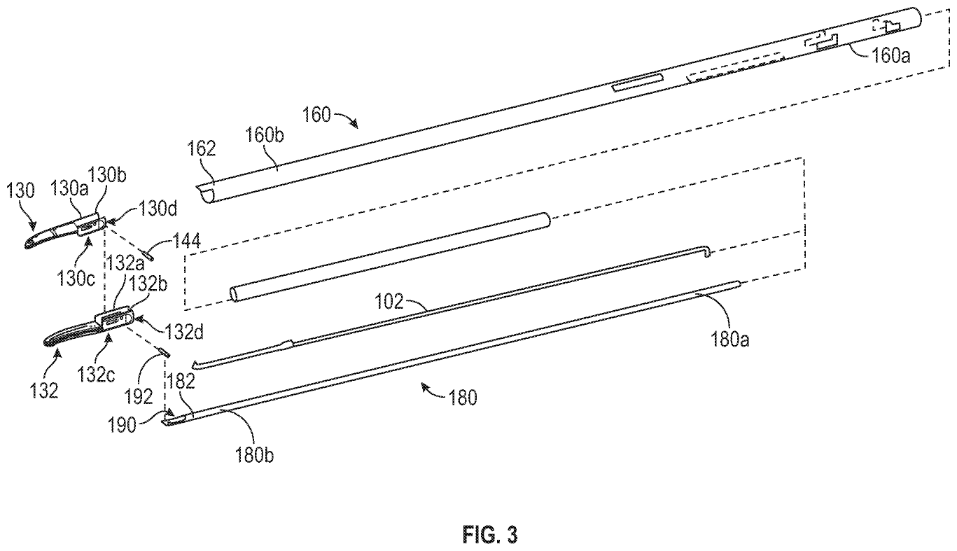

Referring now to FIGS. 2A-3, the upper and lower jaw members 130, 132 of the end effector 114 are electrically coupled to a cable 143, and thus to the generator 141 (e.g., via a respective wire extending through the elongated shaft assembly 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130.

A proximal portion of each of the jaw members 130, 132 includes two laterally spaced parallel flanges or “flags” 130a, 130b, and 132a, 132b respectively, extending proximally from a distal portion of the jaw members 130 and 132. A lateral cam slot 130c and a lateral pivot bore 130d extend through each of the flags 130a, 130b of the upper jaw member 130. Similarly, a lateral cam slot 132c and a lateral pivot bore 132d extend through each of the flags 132a, 132b of the lower jaw member 132. The pivot bores 130d, 132d receive a pivot pin 144 in a slip-fit relation that permits the jaw members 130, 132 to pivot about the pivot pin 144 to move the end effector 114 between the open and closed configurations (FIGS. 2A and 2B, respectively).

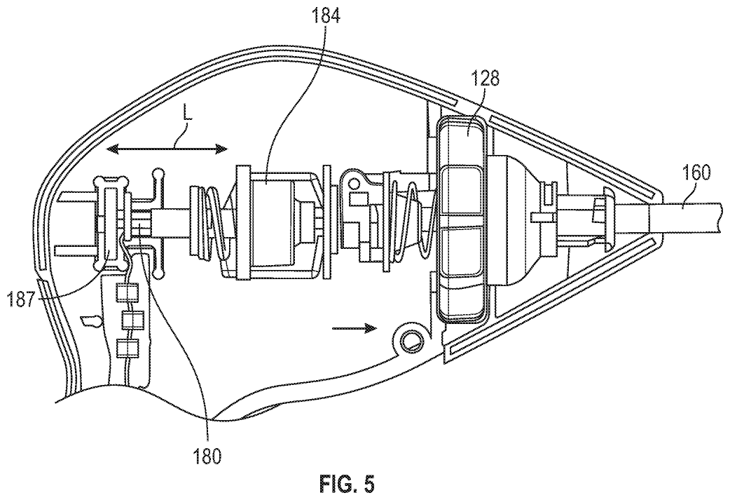

With reference to FIGS. 3-5, the elongated shaft assembly 116 includes various components that operatively couple the end effector 114 to the various actuators supported by the housing 112 (FIG. 1). In particular, the elongated shaft assembly 116 includes an outer shaft 160 and an inner shaft 180 disposed within the outer shaft 160. The outer shaft 160 is configured for longitudinal motion with respect to the inner shaft 180. The outer shaft has a distal end portion 160b having a distal end 162, and a proximal end portion 160b. The proximal end portion 160a of the outer shaft 160 is configured for receipt within the handle housing 112 (FIG. 1), and includes features for operatively coupling the outer shaft 160 to the actuators supported thereon, e.g. the movable handle 122. In particular, the movable handle 122 may be operatively coupled to the outer shaft 160 by a clevis 178 defined at an upper end of the movable handle 122. The clevis 178 extends upwardly about opposing sides of a drive collar 184 (FIG. 5) supported on the outer shaft 160, such that pivotal motion of the movable handle 122 induces corresponding longitudinal motion of the drive collar 184 and, in turn, the outer shaft 160, along the longitudinal axis A-A.

The inner shaft 180 may be a rod, stamped metal, or other suitable mechanical component, and includes a distal end portion 180b having a distal end 182, and a proximal end portion 180a. The distal end portion 180b of the inner shaft 180 defines a longitudinal recess 190 that provides clearance for the pivot pin 144 and thus, permits longitudinal reciprocation of the pivot pin 144 (via longitudinal reciprocation of the outer shaft 160) independent of the inner shaft 180. The proximal end portion 180a of the inner shaft 180 includes a washer 187 coupled thereto. The washer 187 is supported within the distal portion of the housing 112 and serves to prohibit longitudinal motion of the inner actuation member 180 along a longitudinal axis A-A (FIG. 1).

Distally of the longitudinal recess 190 of the inner shaft 180, a cam pin 192 is mechanically coupled (e.g., via welding, friction-fit, laser welding, etc.) to the distal end 182 of the inner shaft 180. The end effector 114 is coupled to the distal end 182 of the inner shaft 180 by the cam pin 192. The cam pin 192 represents a longitudinally stationary reference for the longitudinal movements of the outer shaft 160, the pivot pin 144, and a knife rod 102 (FIG. 3). The cam pin 192 extends through the flags 132a, 132b of the lower jaw member 132 and the flags 130a and 130b of the upper jaw member 130.

Since the inner shaft 180 is coupled to the cam pin 192, when the outer shaft 160 is in the distal position (unactuated) and the inner shaft 180 is in the proximal position relative to the outer shaft 160, the cam pin 192 is located in a proximal position in cam slots 130c and 132c defined through the flags 130a, 130b, 132a, 132b of the jaw members 130, 132, respectively. The outer shaft 160 may be drawn proximally relative to the inner shaft 180 and the cam pin 192 to move the end effector 114 to the closed configuration (see FIG. 2B). Since the longitudinal position of the cam pin 192 is fixed, and since the cam slots 130c, 132c are obliquely arranged with respect to the longitudinal axis A-A, proximal retraction of the outer shaft 160 induces relative distal translation of the cam pin 192 through the cam slots 130c, 132c and jaw member 130 to pivot toward jaw member 132 about the pivot pin 144.

Conversely, when the end effector 114 is in the closed configuration, longitudinal translation of the outer shaft 160 in a distal direction induces relative proximal translation of the cam pin 192 through the cam slots 130c, 132c and jaw member 130 to pivot away from jaw member 132 toward the open configuration. Distal longitudinal motion of the outer shaft member 160 advances jaw member 132 distally such that the cam pin 192 is positioned proximally to pivot jaw member 130 away from jaw member 132 to move the end effector 114 to the open configuration as described above with reference to FIG. 2A.

In alternate embodiments, instead of the inner shaft 180 acting as a stationary reference point for the outer shaft 160, the inner shaft 180 may be axially movable relative to the outer shaft 160 to induce relative motion between the cam pin 192 and the cam slots 130c, 132c of the jaw members 130, 132. In this alternate embodiment, the proximal end portion 180a of the inner shaft 180, rather than the outer shaft 160, is operably coupled to the movable handle 122 via the collar 184.

During manufacturing, various mechanical components are manufactured with a predetermined length that is stored in an RFID (not shown) of the forceps 100. However, in repeating the manufacturing process there may be an inevitable variation in the actual manufactured length. The variance between the predetermined length of the mechanical component and the actual manufactured length of the mechanical component is often based on a normal distribution with a standard deviation based on the manufacturing process. For example, the inner shaft 180 may be manufactured longer or shorter than the predetermined length stored in the RFID resulting in undesired effects in the jaw gap, jaw force, and other aspects of the jaw members 130, 132. In particular, if the inner shaft 180 is manufactured longer than the predetermined length, the gap between the jaw members 130, 132 may be larger than intended, resulting in a decrease in jaw force applied by the jaw members 130, 132 during use of the surgical instrument 100. Likewise, if the inner actuation member 180 is manufactured shorter than the predetermined length, the gap between the jaw members 130, 132 may be smaller than intended, resulting in an increase in jaw force applied by the jaw members 130, 132 during use of the surgical instrument 100.

Similarly, the outer shaft 160 may be manufactured longer or shorter than the predetermined length stored in the RFID resulting in undesired effects similar to those indicated above. In particular, if the outer shaft member 160 is manufactured shorter than the predetermined length, the gap between the jaw members 130, 132 may be larger than intended, resulting in a decrease in jaw force applied by the jaw members 130, 132 during use of the surgical instrument 100. Likewise, if the outer shaft 160 is manufactured longer than the predetermined length, the gap between the jaw members 130, 132 may be smaller than intended, resulting in an increase in jaw force applied by the jaw members 130, 132 during use of the surgical instrument 100.

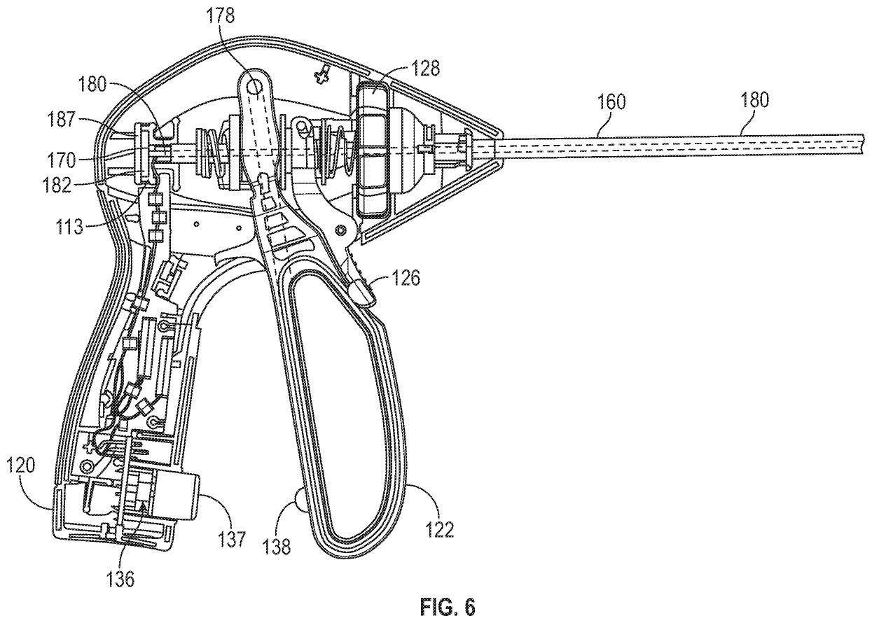

With reference to FIGS. 6-9, in order to account for the natural variance in the dimensions of mechanical components of the surgical instrument 100 (e.g., the lengths of the inner and/or outer shafts 180, 160), the surgical instrument 100 includes a piezoelectric actuator 170 that serves to adjust the length of the associated mechanical component. More specifically, the piezoelectric actuator 170 may be a programmable piezo-based shim configured to alter its shape in response to receiving a signal from the generator 141, whereby the altered shape of the piezoelectric actuator 170 adjusts the length of the associated mechanical component, as will be described in more detail below.

The RFID may be coupled to a memory configured to store a calibration value corresponding to the predetermined length of the various mechanical components, as prescribed by the manufacturer, to account for the natural variance of the mechanical components. The generator 141 may further include a reader (not shown) to interrogate the RFID of the surgical instrument 100. To adjust the mechanical component to the predetermined length, the surgical instrument 100 sends a signal representative of the calibration value to the generator 141 to actuate the piezoelectric actuator 170. When the piezoelectric actuator 170 is actuated, the associated mechanical component of the surgical instrument 100 is moved a selected distance according to the calibration value, corresponding to the difference between the actual manufactured length and the predetermined length stored in the RFID, to adjust a jaw force, a jaw gap, and/or any other suitable combinations of mechanical output.

Referring now to FIG. 6, the piezoelectric actuator 170 may be coupled to or otherwise associated with the inner shaft 180 for selectively adjusting a length of the inner shaft 180. For example, the piezoelectric actuator 170 may be disposed between and coupled to a portion of the housing 112, such as, for example, a collar 113 extending inwardly from an inner surface of the housing 112, and a mechanical ground, such as, for example, the washer 187. The collar 113 accommodates the piezoelectric actuator 170 therein while providing enough space to allow for expansion and contraction of the piezoelectric actuator 170. As such, with the piezoelectric actuator 170 fixed at its proximal end to the washer 187, the collar 113 allows for expansion in the distal direction while providing a distal limit for the piezoelectric actuator 170.

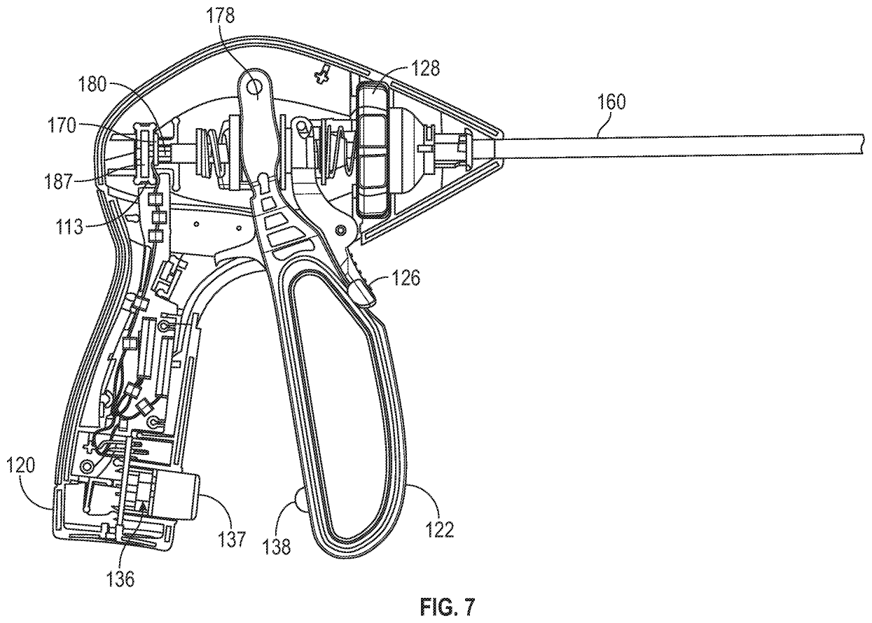

FIG. 7 illustrates another location for coupling the piezoelectric actuator 170 to the inner shaft 180. In particular, the piezoelectric actuator 170 may be electrically coupled in series with the inner shaft 180 and disposed at a location between the proximal and distal end portions 180a, 180b of the inner shaft 180. In this embodiment, the proximal and distal end portions 180a, 180b may be separate components that are joined together via the piezoelectric actuator 170.

To adjust the length of the inner shaft 180, a signal representative of the calibration value of the inner shaft 180 is sent to the generator 141 from the memory in the surgical instrument 100 to actuate the piezoelectric actuator 170. Upon receiving the signal, such as, for example, current, the generator 141 delivers electricity (e.g., voltage or current) to the piezoelectric actuator 170 via electric leads (not shown) disposed on the proximal end portion of the piezoelectric actuator 170. The piezoelectric actuator 170 receives the electricity from the generator 141, which alters the shape and/or size of the piezoelectric actuator 170. For example, the piezoelectric actuator 170 may physically expand or contract, thereby causing a respective increase or decrease in the effective length of the inner shaft 180 along the longitudinal axis A-A. More specifically, when the piezoelectric actuator 170 is disposed between the collar 113 and the washer 187 as shown in FIG. 6, actuation of the piezoelectric actuator 170 axially shifts (e.g. proximally or distally) the inner shaft 180 relative to the outer shaft 160, based on the electricity received by the generator 141. Upon moving the inner shaft 180 axially, the distal end 182 of the inner shaft 180 is repositioned relative to the distal end 162 of the outer shaft 160.

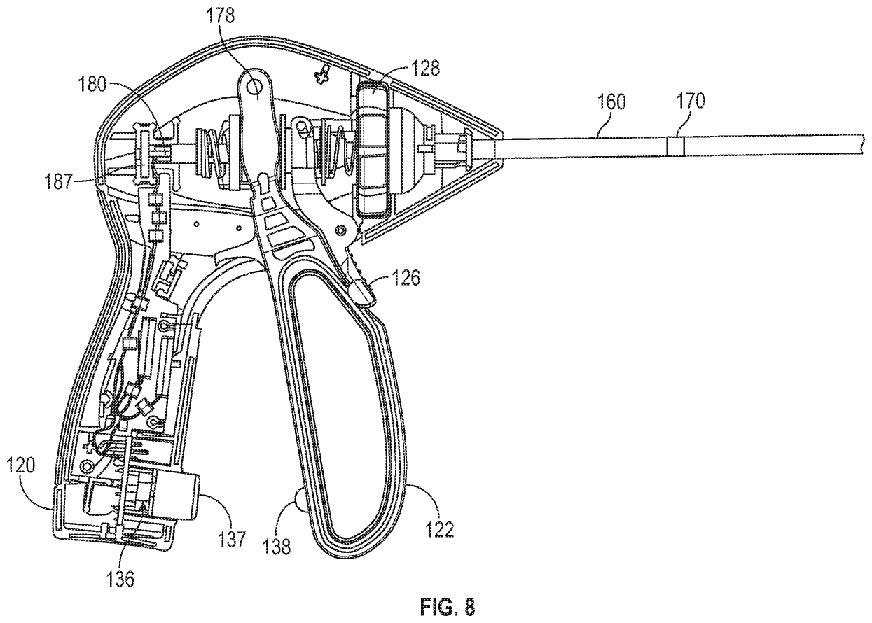

Referring now to FIG. 8, the piezoelectric actuator 170 may be coupled to the outer shaft 160, as opposed to the inner shaft 180. The piezoelectric actuator 170 may be coupled along the outer shaft 160 and between the proximal end portion 160a of the outer shaft 160 and the distal end portion 160b of the outer shaft 160. The piezoelectric actuator 170 being associated with the outer shaft 160 allows for the selective adjustment of an effective overall length of the outer shaft 160 relative to the inner shaft 180 to account for variance in the dimensions (e.g., length) of the outer shaft 160 and/or inner shaft 180.

To adjust the effective length of the outer shaft 160, a signal representative of the calibration value of the outer shaft 160 is sent to the generator 141 from the memory of the surgical instrument 100 to actuate the piezoelectric actuator 170. The generator 141 delivers electricity to the piezoelectric actuator 170 via electric leads based on the calibration value received by the generator 141. Upon the piezoelectric actuator 170 receiving electricity from the generator 141, the piezoelectric actuator 170 alters its shape and/or size to shift the axial location of the distal end 162 of the outer shaft 160 (e.g., proximally or distally) relative to the distal end 182 of the inner shaft 180.

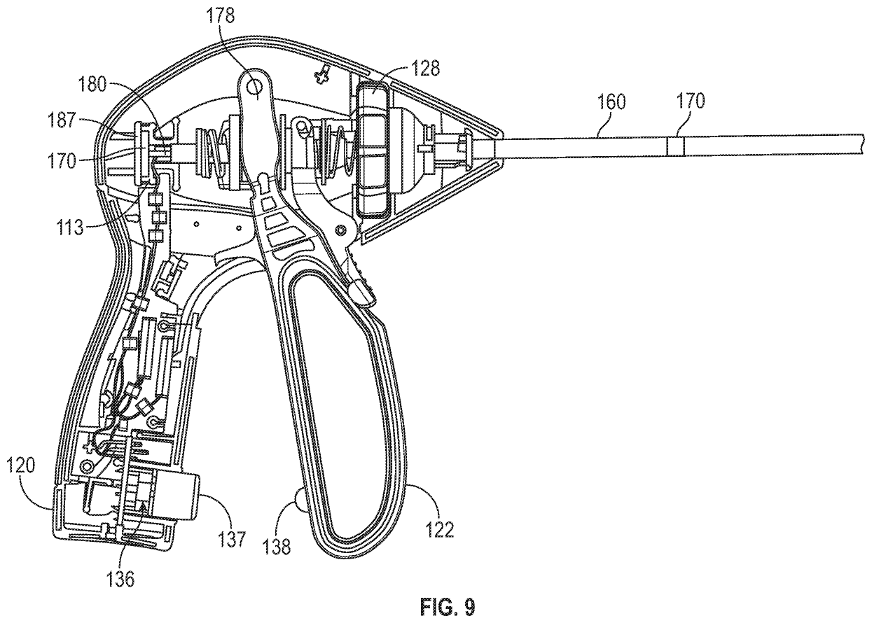

Referring now to FIG. 9, in some embodiments, the surgical instrument 100 may include a plurality of piezoelectric actuators 170 with each coupled to a discrete mechanical component. For example, the piezoelectric actuators 170 may be coupled to or otherwise associated with both the inner shaft 180 and the outer shaft 160. One piezoelectric actuator 170 may be disposed between the washer 187 and the proximal end portion 180a of the inner shaft 180, and another piezoelectric actuator 170 may be disposed in series between the proximal and distal end portions 160a, 160b of the outer shaft 160. The piezoelectric actuators 170 work in tandem to account for variance in the inner shaft 180, the outer shaft 160, and any variation in the jaw gap, jaw force, and/or other aspects of the jaw members 130, 132.

The generator 141 may adjust the overall effective lengths of the inner shaft 180, the outer shaft 160, or both to account for shorter or larger jaw gap, and/or a higher or lower jaw force between the pair of opposed jaw members 130, 132 by delivering electricity to one or both of the piezoelectric actuators 170. Actuating the piezoelectric actuators 170 alters their shape and/or size, thereby advancing or retracting the inner shaft 180, the outer shaft 160, or both, along the longitudinal axis A-A to adjust the effective length of the inner shaft 180, the outer shaft 160, or both.

Supplying electricity to the piezoelectric actuator 170 by the generator 141 may be controlled by microcontrollers or integrated circuits over communication protocol embedded in surgical instrument 100, such as, for example I²C, CAN, SPI or 1-Wire serial communication interfaces. The microcontrollers (MCU) may be configured to enable communications with the piezoelectric actuator 170 or a sensor management to detect jaw position, jaw force, temperature, pressure, or light based seal completion. To deliver power to the embedded microcontrollers, cable 143 may include a microcontroller output voltage suitable for powering the embedded microcontrollers, such as, for example voltage output of 5V, and controlled by the generator 141.

An analog or digital signal, such as, for example, a pulse-width modulated (“PWM”), may be applied to the command to, for example, eliminate noise from communication which could alter the signal between the generator 141 and the surgical instrument 100 or allow the use of more than one piezoelectric actuator 170 within surgical instrument 100 each being sufficiently powered by an individual power source. Furthermore, more than one piezoelectric actuator 170 may be individually powered by the individual power source. To deliver power from the generator 141 to actuate the piezoelectric actuator 170, the cable 143 includes a piezoelectric output voltage suitable for driving an excitation voltage of the piezoelectric actuator 170, such as, for example, a voltage output of 12V, 24V, or 48V.

In some embodiments, the PWM signal may occur within the generator 141. The generator 141 is further coupled to a circuit (e.g., similar to an audio line balancer op amp) configured to remove noise between the generator 141 and the surgical instrument 100. The PWM signal occurring within the generator 141 may further include one or more signal wires (e.g., a signal wire and an inverted signal wire) each configured to carry independent signals, with or without the noise filtering. Delivery of electricity from the generator 141 is controlled by the PWM signal of the piezoelectric output voltage delivered through the cable 143.

In some embodiments, the RFID may store calibration values corresponding to optimal mechanical outputs. For example, in one instance, a short circuit can occur during the seal cycle resulting in a regrasp alarm. Short circuit is detected, for example, when impedance is below a low impedance threshold and/or a phase is above an upper threshold. To reduce the occurrence of the short circuit and the subsequent regrasp alarm, the generator 141 receives a calibration value, from the RFID, to actuate the piezoelectric actuator 170 to adjust the jaw gap to an optimal output based on the calibration value.

In the event that the insulator of one or both of the jaw members 130, 132 malfunctions, the stored calibration value of the RFID may be updated to store a new calibration value to account for the malfunctioning insulator or insulators. The generator 141 receives the updated calibration value to actuate the piezoelectric actuator 170 to adjust the jaw gap and, in effect, self-heal from the malfunctioning insulator or insulators. The RFID may be further programmed to store a calibration value that can negate the need for the insulator of the jaw members 130, 132 enabling grasping of fine tissue. As noted above, the malfunction of even one insulator may result in the sealing plates 148 and 150 having a direct electrical short. In updating the calibration value to negate the need for the insulator, the electrical short is detected by the generator 141 and in response to the detected electrical short the calibration value is updated and sent to the generator 141 to actuate the piezoelectric actuator 170 to adjust the jaw members 130, 132 to operate without the insulator. Negating the need of insulators may reduce the cost of manufacturing the sealing plates 148 and 150, allow for more flexible design of the contacting surface area between the jaw member 130, 132, and more flexible design of the proximal end bias or distal tip bias of the jaw members 130, 132. Additionally other modifications may be made during manufacturing necessary for tissue sealing, such as, for example, negating the need for limits with respect to the high point load stresses for tissues and/or providing more bias at the proximal end or distal end of the jaw member 130, 132.

In other instances, for example, not completing the seal cycle before a predetermined timeout period causes a timeout alarm to be issued by the generator 141 and/or the surgical instrument 100. To reduce the occurrence of extended seal cycles and resulting timeout alarms, the generator 141 receives a calibration value from the RFID, to actuate the piezoelectric actuator 170 to adjust the jaw gap and jaw force to an optimal output necessary to stimulate a sealing event. In the event that completing the seal cycle takes longer than the predetermined timeout period, the calibration value of the RFID may be updated to store a new calibration value to account for the duration of the seal cycle. In updating the calibration value, the generator 141 monitors an impedance and in response to the detected impedance the calibration value is updated and sent to the generator 141 to actuate the piezoelectric actuator 170 during or after the seal cycle to adjust the jaw force, jaw gap, and other aspects of the jaw members 130, 132 to achieve a targeted exit condition.

For example, determining and updating the calibration value may be implemented through machine learning. Machine learning may be used to determine the calibration value during manufacturing. The machine learning may be further developed to include an algorithm used to iterate the calibration value by actuating the movable handle 122 to physically open and close the jaws members 130, 132 to achieve tighter standards of deviation for the various combinations of mechanical outputs. Each interation of the calibration value would tweak, between individual sealing cycles and within individual sealing cycles, various combinations of the mechanical outputs to reduce overall time to seal, learn surgeon's habits, patient tissue types, procedures performed and other various conditions necessary to optimize the user's experience with operating the surgical instrument 100.

In another aspect of the present disclosure, rather than coupling the piezoelectric actuator 170 to the inner shaft 180 and/or the outer shaft 160, the piezoelectric actuator 170 may be used in a “fly by wire” design. In the “fly by wire” design, the piezoelectric actuator 170 is configured to alter its shape sufficiently enough to equal the full range of mechanical travel of the inner shaft 180 and/or the outer shaft 160 to open and close the jaw and further scaled through various mechanical solutions such as, for example, linkages and levers. Typically, other devices similar to surgical instrument 100 include means of determining position of the movable handle 122 such as, for example, a potentiometer, optical distance measurement, encoder graphic or decal, applied to the movable handle 122. The “fly by wire” design includes software configured to process the actuation of the movable handle 122 to alter the rate or range of the physical response of the piezoelectric actuator 170. The software processes the actuation of the movable handle 122 and scales the output motor range to create a “fine dissection mode” where the jaw gap and the rate of motion is limited while allowing more generous motion with the handle 122. Once the actuation of the movable handle 122 is processed by the software, the software sends a signal to the piezoelectric actuator 170 to adjust the position of the piezoelectric actuator 170 to correspond to the position of the movable handle 122. The force generated by actuating the movable handle 122 is independent from the input force, resulting in the appearance of light force, precision of motion, and reduced fatigue while generating significant loads for grasping the tissue between the jaw members 130, 132.

The software may further process the actuation of movable handle 122 to account for the non-linearity of jaw motion and jaw force due to the use of the mechanical solutions. Non-linearity in the jaw motion and jaw force can include, for example, a single point accelerating or decelerating relative to other points during the course of the jaw closure resulting in a twitchy mechanism travel or a single point not properly changing from maximum to minimum aperture or vice versa, throughout the mechanism travel as found in existing forceps.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical instrument, comprising:
a handle housing;
a handle operably coupled to the handle housing;
an outer shaft extending distally from the handle housing;
an inner shaft axially disposed within the outer shaft and including a cam pin mechanically coupled to a distal end portion of the inner shaft, at least one of the outer shaft or the inner shaft being selectively movable relative to the other along a longitudinal axis in response to an actuation of the handle; and
a piezoelectric actuator coupled to the outer shaft or the inner shaft, wherein the piezoelectric actuator is configured to adjust a distance between a distal end of the outer shaft and a distal end of the inner shaft.

2. The surgical instrument according to claim 1, wherein the piezoelectric actuator is a programmable piezo-based shim.

3. The surgical instrument according to claim 1, wherein the piezoelectric actuator is disposed between a proximal end of the inner shaft and a portion of the housing, such that actuation of the piezoelectric actuator adjusts an axial location of the proximal end of the inner shaft relative to the portion of the housing.

4. The surgical instrument according to claim 3, wherein the proximal end of the inner shaft is fixed to the piezoelectric actuator and the outer shaft is configured to move relative to the inner shaft along the longitudinal axis in response to actuation of the handle.

5. The surgical instrument according to claim 1, wherein the piezoelectric actuator is disposed between and interconnects a proximal end portion of the inner shaft and the distal end portion of the inner shaft.

6. The surgical instrument according to claim 1, wherein the piezoelectric actuator is disposed between and interconnects a proximal end portion of the outer shaft and a distal end portion of the outer shaft.

7. The surgical instrument according to claim 1, further comprising an end effector including a pair of opposing first and second jaw members operably coupled about a common pivot such that at least one of the jaw members is movable relative to the other jaw member from a first position in which the jaw members are disposed in spaced relation to one another to a second position, in which the jaw members cooperate to grasp tissue therebetween, at least one of the first and second jaw members defining a camming slot configured to engage the cam pin to move the at least one movable jaw member between the first position and the second position upon relative longitudinal movement between the inner and outer shafts.

8. The surgical instrument according to claim 7, further comprising a switch supported by the handle housing and configured to be engaged by the handle to initiate delivery of electrosurgical energy from an electrosurgical energy source to the end effector to treat tissue.

9. A method of calibrating a surgical instrument, the method comprising:
sending a signal representative of a calibration value from the surgical instrument to a generator that is electromechanically coupled to the surgical instrument; and
causing a piezoelectric actuator disposed within the surgical instrument to move a mechanical component of the surgical instrument a distance corresponding to the calibration value to adjust a mechanical output of the surgical instrument.

10. The method according to claim 9, wherein the mechanical component of the surgical instrument is an inner shaft or an outer shaft, at least one of the outer shaft or the inner shaft being selectively movable relative to the other in response to actuation of a handle of the surgical instrument to move an end effector between an open and closed configuration.

11. The method according to claim 10, wherein moving the mechanical component includes changing a distance between a distal end of the outer shaft and a distal end of the inner shaft.

12. The method according to claim 11, wherein the piezoelectric actuator is coupled to a proximal end of the inner shaft, such that the actuation of the piezoelectric actuator adjusts an axial location of the proximal end of the inner shaft.

13. The method according to claim 10, wherein the mechanical output is a force applied by the end effector to tissue upon moving the end effector to the closed configuration.

14. The method according to claim 10, wherein the mechanical output is a gap defined between first and second jaw members of the end effector upon moving the end effector to the closed configuration.

15. The method according to claim 10, further comprising:
detecting an electrical short between jaw members of the end effector; and
actuating the piezoelectric actuator to increase a gap defined between the jaw members in response to detecting the electrical short.

16. The method according to claim 9, wherein actuating the piezoelectric actuator includes delivering electricity from the generator to the piezoelectric actuator to alter a shape of the piezoelectric actuator.

17. An electrosurgical system for performing electrosurgery, comprising:
an electrosurgical generator configured to provide electrosurgical energy; and
an electrosurgical instrument including:
a handle housing;
a handle operably coupled to the handle housing;
an outer shaft extending distally from the handle housing;
an inner shaft axially disposed within the outer shaft, at least one of the outer shaft or the inner shaft being selectively movable relative to the other along a longitudinal axis in response to actuation of the handle; and
a piezoelectric actuator coupled to the outer shaft or the inner shaft and in electrical communication with the generator, wherein the piezoelectric actuator is configured to adjust a distance between a distal end of the outer shaft and a distal end of the inner shaft in response to an electrical signal received from the generator.

18. The electrosurgical system according to claim 17, wherein the piezoelectric actuator is disposed between a proximal end of the inner shaft and a portion of the housing, such that an actuation of the piezoelectric actuator adjusts an axial location of the proximal end of the inner shaft relative to the portion of the housing.

19. The electrosurgical system according to claim 18, wherein the proximal end of the inner shaft is fixed to the piezoelectric actuator and the outer shaft is configured to move relative to the inner shaft along the longitudinal axis in response to actuation of the handle.

20. The electrosurgical system according to claim 17, wherein the surgical instrument includes a pair of opposing first and second jaw members operably coupled about a common pivot and configured to move between open and closed configurations in response to relative longitudinal movement between the inner and outer shafts, the actuation of the piezoelectric actuator adjusting at least one of a force applied to tissue disposed between the pair of first and second jaw members or a gap defined between the pair of first and second jaw members.
